(19) United States
(12) Patent Application Publication
(10) Pub. No.: US 2012/0121721 A1
(43) Pub. Date: May 17, 2012

(54) SKIN CARE PRODUCTS AND COMPOSITIONS

(76) Inventor: Michael James, Laguna Beach, CA (US)

(21) Appl. No.: 12/947,716

(22) Filed: Nov. 16, 2010

(51) Int. Cl. A61K 36/9066; A61K 36/82; A61K 36/889; A61K 36/16; A61K 36/45; A61K 35/64; A61P 35/00; A61P 17/00; A61P 17/02; A61P 17/06; A61P 17/10; A61P 29/00; A61P 25/28; A61P 19/02; A61P 31/22; A61K 36/886

(52) U.S. Cl. 424/537; 424/756; 424/744; 424/729; 424/727; 424/752; 424/732

(57) ABSTRACT

Disclosed herein is a composition for treating maladies in humans or animals that includes from about 0.01% to about 40% by weight of a member of the curcuma genus, from about 0.01% to about 40% by weight of a member of the coffea genus and the balance being a pharmaceutically acceptable carrier.

### FIELD OF THE INVENTION

The present invention relates to skin care or dermatological products and compositions that have beneficial effects on the skin and/or the user's health.

### BACKGROUND OF THE INVENTION

Skin care products and compositions are used for achieving healthy and attractive skin, blocking harmful UV rays and healing or alleviating dermatological ailments or disorders. The present invention is directed to skin care products and treatments that enhance or improve the effects of prior skin care products and improving other aspects of a user's health.

### SUMMARY OF THE PREFERRED EMBODIMENTS

In accordance with a first aspect of the present invention there is provided a composition that includes from about 0.01% to about 40% by weight of a member of the curcuma genus, from about 0.01% to about 40% by weight of a member of the coffea genus and the balance being a pharmaceutically acceptable carrier. In a preferred embodiment, the member of the curcuma genus is turmeric and the member of the coffea genus is coffea arabica. Application of the composition to the skin of an animal improves may improve skin cancer and pre-cancers, hemorrhoids, rashes, wounds, scars, age spots, polyps, bedsores, saddle sores, hot spots, insect bites, shingles, herpes, eczema, and psoriasis, among other maladies. In a preferred embodiment, the composition includes from about 0.1% to about 50% noni juice (Citrifolla Morinda), and/or about 0.1% to about 50% aloe vera juice (Aloe barbadensis).

In another preferred embodiment, the composition includes from about 0.001% to about 5% by weight gingko biloba. In another preferred embodiment, the composition includes from about 0.01% to about 40% of a Camellia sinensis tea, such as white tea, yellow tea, green tea, black tea, oolong tea or Pu-erh tea. In a preferred embodiment, the composition further includes from about 0.1% to about 50% of one or more oils, such as grapeseed oil (Vitis vinifera), jojoba oil (Simmondsia chinensis), coconut oil (Cocos nucifera), neem oil (Azadirachta indica), kukui nut oil (Aleurites moluccana), macadamia nut oil (Macadamia integrifolia).

In another preferred embodiment, the composition further includes from about 0.1% to about 50% of one or more fruit juices, such as acai juice (Euterpe oleracea), blueberry juice (any in the Vaccinium genus), cranberry juice (Vaccinium oxycoccos), mangosteen juice (Garcinia mangostana), pomegranate juice (Punica granatum). In a preferred embodiment, the composition includes from about 0.1% to about 60% honey. In a preferred embodiment, the composition can include a UV blocking agent, a moisturizing agent, an enhancer, or a skin care agent.

In accordance with another aspect of the present invention, there is provided a method of treating a condition selected from the group consisting of dry skin, dandruff, warts, acne, sebaceous cysts, wounds, rashes, shingles, herpes/cold sores, boils, pimples, polyps, skin cancers, hemorrhoids, scars, psoriasis, eczema, pruritus, age spots, reduced skin moisture, spider veins, senile purpura, lentigines, melasmas, deepening of skin lines, blotches, wrinkles, blemished skin, nodules, atrophy, rosacea, impetigo, actinic keratosis precancerous lesions, telangiecatic skin, hyperpigmented skin, hyperkeratotic skin, nail infections, inflammatory dermatoses, tumors, cancers, Alzheimer's disease, arthritis and lupus. The method includes the step of administering to a human or animal a composition comprising from about 0.01% to about 40% by weight of a member of the curcuma genus, from about 0.01% to about 40% by weight of a member of the coffea genus and the balance being a pharmaceutically acceptable carrier.

The invention, together with additional features and advantages thereof, may be best understood by reference to the following description.

### DETAILED DESCRIPTION OF THE PREFERRED EMBODIMENTS

A dermatological/skin care or pharmaceutical composition that advantageously manages dermatological and other conditions is described herein. The compositions can be administered topically, orally, rectally, by injection or the like. The skin care composition may be used alone or in conjunction with another composition, such as sunscreen, to manage dermatological conditions. In a preferred embodiment, the composition includes at least a member of the coffea genus, preferably coffee arabica, and a member of the curcuma genus, preferably turmeric extract. In a preferred embodiment, the turmeric extract is in the form of a powder. The invention also includes methods of administering a therapeutically effective amount of the skin care composition for management of dermatological conditions.

The terms “managing” or “management,” as used herein, include one or more of the prevention, treatment, or modification of a dermatological condition. Preferably the terms “managing” or “management,” as used herein, include one or more of treatment, or modification of a dermatological condition.

The term “dermatological conditions,” as used herein, means conditions present anywhere on the skin caused by aging or extrinsic factors such as sunlight, radiations, air pollution, wind, cold, dampness, heat, chemicals, smoke, and smoking. Dermatological conditions include, but are not limited to, dry skin, dandruff, warts, acne, keratosis, sebaceous cysts, wounds, rashes, shingles, herpes/cold sores, boils, pimples, polyps, skin cancers, hemorrhoids, scars, psoriasis, eczema, pruritus, age spots, reduced skin moisture, spider veins, senile purpura, lentigines, melasmas, deepening of skin lines, swelling, bumps or bruises on the skin, blotches, wrinkles, blemished skin, nodules, atrophy, rosacea, impetigo, actinic keratosis precancerous lesions, elastotic changes characterized by leathery, course, rough, dry and yellowish skin, telangiecatic skin, hyperpigmented skin, hyperkeratotic skin, nail infections, inflammatory dermatoses, and damage to hair including, but not limited to, hair breakage, weathering damage, and thinning of hair, chicken pox, measles, flu, common cold, swollen lymph nodes, mosquito borne illnesses, skin inflammation due to chemical exposure, poisonous plant rashes.

In another preferred embodiment, the composition can be in the form of a pill, tonic, syrup, capsule or other form that can be taken or administered orally and/or internally. The oral composition can be used to treat conditions such as tumors, cancers, Alzheimer's disease, arthritis, lupus, vitiligo or any of those listed above.

In another preferred embodiment, the composition can be in the form of a suppository, cream or other treatment for rectal administration to treat, hemorrhoids, polyps and other rectal or colon issues. It will be understood that none of the treatment lists herein are intended by limiting.

A preferred embodiment of the composition includes turmeric powder (Curcuma Longa or others in the Curcuma genus) from about 0.01% to about 40% by weight, preferably from about 1% to about 20% and most preferably from about 2% to about 15%, and coffee (Coffea Arabica or others in the coffea genus) from about 0.01% to about 40%, preferably from about 1% to about 25% and most preferably from about 2% to about 20%. This composition can be used to treat skin conditions in humans and other mammals. The conditions can include, but are not limited to skin cancer and pre-cancers, hemorrhoids, rashes, wounds, scars, age spots, polyps, bedsores, saddle sores, hot spots, insect bites, eczema, and psoriasis.

In a preferred embodiment, the composition includes noni juice (Citrifolla Morinda) from about 0.1% to about 35%, preferably from about 1% to about 40% and most preferably from about 4% to about 35%, and aloe vera juice (Aloe barbadensis) from about 0.1% to about 50%, preferably about 1% to about 40% and most preferably from about 4% to about 35%. This composition can be used to speed the treatments listed above and internal tumors and cancers.

In a preferred embodiment, the composition includes gingko biloba from about 0.001% to about 5%, preferably about 0.1% to about 3% and most preferably from about 0.2% to about 2%.

In a preferred embodiment, the composition includes one or more Camellia sinensis teas (white tea, yellow tea, green tea, black tea, oolong tea or Pu-erh tea) from about 0.01% to about 40%, preferably from about 1% to about 25% and most preferably from about 2% to about 20%.

In a preferred embodiment, the composition includes the use of instant coffee and/or instant tea instead of brewed coffee and tea from about 0.01% to about 40%, preferably from about 1% to about 25% and most preferably from about 2% to about 20%.

In a preferred embodiment, the composition includes one or more of the following oils from about 0.1% to about 50%, preferably from about 1% to about 40% and most preferably from about 4% to about 35%: grapeseed oil (Vitis vinifera), jojoba oil (Simmondsia chinensis), coconut oil (Cocos nucifera), neem oil (Azadirachta indica), Kukui nut oil (Aleurites moluccana), Macadamia nut oil (Macadamia integrifolia).

In a preferred embodiment, the composition includes one or more of the following juices from about 0.1% to about 50%, preferably from about 1% to about 40% and most preferably from about 4% to about 75%: acai juice (Euterpe oleracea), blueberry juice (any in the Vaccinium genus), cranberry juice (Vaccinium oxycoccos), mangosteen juice (Garcinia mangostana), pomegranate juice (Punica granatum).

In a preferred embodiment, the composition includes honey from about 0.1% to about 60%, preferably from about 1% to about 50% and most preferably from about 5% to about 45%.

It will be understood that any form of the various ingredients discussed herein can be used. For example, concentrates or extracts of any or all ingredients may be used. It will be further understood that the composition can include or be co-administered with a medicine either orally or topically, or rectally, depending on the condition being treated or managed. For example, fluorouracil cream (Carac) used to treat skin cancer and pre-cancers may cause irritation and redness of the skin and/or pain. However, the inventive composition helps reduce these side effects and therefore may be combined with the fluorouracil cream of other irritant. The composition can also include an antibiotic.

It will also be understood that the composition can be administered orally either in pure form, diluted with water, juice or a combination of juices, alcoholic or non-alcoholic beverages. The composition can also be administered topically with a spray, gel, cream, salve, or lotion either in pure form or diluted with ingredients such as, but not limited to, water, oil, alcohol, or combinations of those and other ingredients known to a person skilled in the art. The composition can also be administered topically with an adhesive skin patch or bandage or rectally as a suppository, as an additive to a colonic treatment, or in an enema. The composition can be used as an insect repellant. Neem oil can be added to the composition to aid in repelling insects. In an embodiment where the composition includes aloe vera, the composition can be used as a shaving gel, cream or foam. In another embodiment, the composition can be used as a shaving gel, cream or foam without aloe vera.

In a preferred embodiment, the composition includes zinc oxide, and/or 20% zinc oxide ointment, or titanium oxide, or any other sunscreen compositions, lotions, creams, or ointments commonly used for topical use as a sunscreen.

The skin care composition can be in the form of a cream, gel, oil, spray, powder, paste, clay or any other form, way or method known in the art for administering the composition to the skin or a subject whether human or animal.

In preferred embodiments the skin care or oral composition includes the following exemplary formulations: 1) coffee, green tea, honey grape seed oil and turmeric powder; 2) coffee, green tea, grape seed oil, turmeric powder, coconut oil, noni juice, aloe vera juice, and honey; 3) noni juice, aloe vera juice, grapeseed oil, jojoba oil, neem oil, pomegranate juice, cranberry juice, blueberry juice, instant coffee (e.g., Folgers® Crystals), instant tea (preferably green tea, but may use black or other tea), coconut oil, honey, ground turmeric, zinc oxide powder, ginkgo biloba and zinc oxide ointment; 4) noni juice, aloe vera juice, grapeseed oil, jojoba oil, coconut oil, honey, coffee, green tea, ground turmeric, zinc oxide powder, ginkgo biloba and zinc oxide ointment; 5) turmeric and coffee; and 6) turmeric, coffee, noni juice and aloe vera. These lists are in no way intended to be limiting.

In other preferred embodiments, the skin care or oral composition includes the following exemplary embodiments: Formulation A includes 2 ounces of brewed coffee, 2 ounces of brewed green tea, 1 tablespoon of honey, 2 ounces of grapeseed oil, and 3 ounces of turmeric powder. Formulation B includes 2 ounces of brewed coffee, 2 ounces of brewed green tea, 3 ounces of turmeric powder, 2 ounces of grapeseed oil, 2 ounces of coconut oil, 2 ounces of noni juice, 2 ounces of aloe vera juice and 3 ounces of honey. Formulation C includes 2 ounces of instant coffee, 2 ounces of instant tea, 2 ounces of noni juice, 2 ounces of aloe vera juice, 1 ounce of grapeseed oil, 1 ounce of coconut oil, 1 ounce of jojoba oil, 4 ounces of honey. 1 ounce of these ingredients mixed together is then mixed with 1 teaspoon of turmeric powder, 240 mg gingko biloba and 2 ounces of zinc oxide ointment. Formulation D includes 2 ounces of noni juice, 2 ounces of aloe vera juice, 2 ounces of instant coffee, 2 ounces of instant tea, 4 ounces of honey, 1 ounce of grapeseed oil, 1 ounce of jojoba oil, 1 ounce of coconut oil, 1 ounce of neem oil, 1 ounce of pomegranate juice, 1 ounce of cranberry juice, and 1 ounce of blueberry juice. 1 ounce of these ingredients mixed together is then mixed with 1 teaspoon of turmeric powder, 240 mg. gingko biloba, ½ teaspoon of zinc oxide powder and 2 ounces of zinc oxide ointment. Formulation E includes 1 ounce of grapeseed oil, 1 ounce of jojoba oil, 1½ ounce of turmeric powder, 720 mg. ginkgo biloba powder, 2 ounces of instant coffee and 2 ounces of instant tea. The turmeric powder, ginkgo biloba powder, instant coffee and instant tea are dissolved in the oils at an elevated temperature and then the following is added: 2 ounces of noni juice, 2 ounces of aloe vera juice, and 4 ounces of honey. Formulation F includes ½ ounce of the Formulation E above with ½ ounce of pomegranate juice, ½ ounce of cranberry juice, and ½ ounce of blueberry juice. Formulation G is an oral version using the ingredients of Formula C, but without the zinc oxide ointment, and includes 2 ounces of instant coffee, 2 ounces of instant tea, 2 ounces of noni juice, 2 ounces of aloe vera juice, 1 ounce of grapeseed oil, 1 ounce of coconut oil, 1 ounce of jojoba oil, 4 ounces of honey, 1½ ounces of turmeric powder and 720 mg. ginkgo biloba.

Mixing the powders into the oils can also be used with Formulations C and D to make Formulations H and I. Formulation J includes any one of Formulations A, B, C, D, or E mixed with aloe vera gel instead of zinc oxide ointment to make a topical application for sunscreen or for medicinal absorption through the skin.

Any fruit extract is suitable for use in the composition and methods of the invention. For example, fruit extracts may be obtained from apricots, apples, blueberries, pears, peaches, pineapples, papayas, cherries, kiwis, pomegranates, tangerines, oranges, grapes, or a combination thereof. The fruit extract may be obtained from any part of the plant including, for example, the fruit, the skin or rind of the fruit, the seeds, the bark, the leaves, the roots, or the stem.

Any suitable pharmaceutically acceptable carrier may be used with the compositions taught herein, as will be readily apparent to one of ordinary skill in the art. Pharmaceutically acceptable carriers include, but are not limited to, water, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproply methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones. For example, preferred topical formulations of the pharmaceutical composition may include a silicon-containing carrier, preferably a silicone, but in amounts insufficient to cause substantial irritation. Suitable silicones include cyclomethicone or a mixture of cyclopentasiloxane and dimethicone/vinyldimethicone crosspolymer.

In a preferred embodiment of the skin care composition, the composition preferably includes one or more moisturizing agents. “Moisturizing agent,” as used herein, is used to include any agent that facilitates hydration of the skin by inhibiting or preventing loss of water from the skin, absorbs water from the atmosphere and hydrates the skin, or enhances the skin's own ability to absorb water directly from the atmosphere, or a combination thereof. Moisturizing agents generally minimize or prevent the skin from drying and cracking. Cracked skin is more susceptible to environmental factors that generate free radicals, which is believed to cause further damage to the skin. Thus, moisturizing or facilitating moisturizing skin reduces damage from free radicals and can help manage many dermatological conditions. Suitable moisturizing agents include, but are not limited to, acidic components, hydrophobic agents, and hydrophilic agents, or combinations thereof. Moisturizers, when used, are typically present in an amount from about 0.01 to 20 weight percent, preferably about 0.05 to 10 weight percent, more preferably from about 0.1 to 1 weight percent of the composition.

As used herein, the term “enhancer” refers to a substance that can enhance the beneficial effect of at least one skin care ingredient co-administered or co-applied with the substance. The skin care compositions of the present invention may comprise enhancers selected from the group consisting of cyclodextrin, pentasodium pentetate, phytic acid, potassium citrate, sodium citrate, potassium gluconate and sodium gluconate, with the optional inclusion of at least one optional enhancer selected from the group consisting of ethylene diaminetetraacetic acid (EDTA), EDTA disodium salt, EDTA trisodium salt and EDTA tetrasodium salt. For instance, the skin care product of the present invention comprises at least two enhancers selected from the group consisting of cyclodextrin, pentasodium pentetate, phytic acid, potassium citrate and potassium gluconate, with the optional inclusion of at least one of EDTA, EDTA disodium salt, EDTA trisodium salt and EDTA tetrasodium salt. Preferably, when the skin care product includes citrate as among the at least two enhancers, either potassium citrate or sodium citrate, but not both, is used. Similarly, when the skin care product includes gluconate as among the at least two enhancers, either potassium gluconate or sodium gluconate, but not both, is used.

Suitable emollient/humectant/moisturizer that can be used in the compositions of the invention include the emullients and moisturizing agents disclosed below. Examples of emullients that can be used in the skin moisturizing products of the invention are glycerin, cetyl alcohol, cetearyl isononanoate, cetearyl octanoate, decyl oleate, isooctyl stearate, coco caprylate/caprate, ethylhexyl hydroxystearate, ethylhexyl isononanoate, isoproyl palmitate, isopropyl isostearate, isopropyl myristate, oleyl oleate, hexyl laurate, paraffinum liquidum, PEG-75 lanolin, PEG-7 glyceryl cocoate, petrolatum, ozokerite, cyclomethicone, dimethicone, dimethicone copolyol, dicaprylyl ether, butyrospermum parkii, buxus chinensis, canola, carnauba cera, copernicia cerifera, oenothera biennis, elaeis guineensis, prunus dulcis, squalane, zea mays, glycine soja, helianthus annuus, lanolin, hydrogenated castor oil, hydrogenated coconut oil, avocodo oil, hydrogenated polyisobutene, sucrose cocoate, stearoxy dimethicone, lanolin alcohol, isohexadecane. Examples of moisturizing agents that can be used are butylene glycol, cetyl alcohol, dimethicone, dimyristyl tartrate, glucose, glycereth-26, glycerin, glyceryl stearate, hydrolyzed milk protein, lactic acid, lactose and other sugars, laureth-8, lecithin, octoxyglycerin, PEG-12, PEG-135, PEG-150, PEG-20, PEG-8, pentylene glycol, hexylene glycol, phytantriol, polyquaternium-39, PPG-20 methyl glucose ether, propylene glycol, sodium hyaluronate, sodium lactate, sodium PCA (sodium salt of 1-pyrrolidone carboxylic acid), sorbitol, succinoglycan, synthetic beeswax, tri-C14 15 alkyl citrate, starch.

As used herein, the term “anti-acne agent” means a substance that has a beneficial effect in treating or preventing acne. Suitable anti-acne agents can be drying agents, keratolytic agents, epidermolytic agents, antimicrobial agents and retinoids. Examples of anti-acne agents include sulfur, resorcinol, glycolic acid, lactic acid, pyruvic acid, salicylic acid, retinoic acid, derivatives of retinoic acid, and antimicrobial agents, e.g., farnesol, benzoyl peroxide, erythromycin, triclosan, azelaic acid, clindamycin, chlorhexidine, neomycin, miconazole, clotrimazole and tetracycline. Suitable anti-acne agents include salicylic acid, 5-octanoyl salicylic acid, resorcinol, retinoids such as retinoic acid and its derivatives, sulfur-containing D and L amino acids other than cysteine, lipoic acid, antibiotics and antimicrobials such as benzoyl peroxide, octopirox, tetracycline, 2,4,4′-trichloro-T-hydroxydiphenyl ether, 3,4,4′-trichlorobanilide, azelaic acid, phenoxyethanol, phenoxypropanol, phenoxisopropanol, ethyl acetate, clindamycin and melclocycline, flavonoids, and bile salts such as scymnol sulfate, deoxycholate and cholate.

The term “skin care agent” refers to an agent that has one or more beneficial effects on the care and/or hygiene of the skin. The skin care agent can be selected from the group consisting of antioxidants, free-radical scavengers, antimicrobial agents, anti-acne agents, reducing agents, vitamins, skin protecting agents, skin bleaching agents, skin conditioning agents, skin soothing agents, skin healing agents, collagen promoters, exfoliators, self tanners, chelators, hair removers, anti-erythema agents, anti-redness agents, anti-rosacea agents, depuffing agents, anti-edema agents, anti-swelling agents, hyaluronic acid, green tea extract, P. emblica (Amla), arnica, chamomile extract and cucumber extract. The skin care agent is, preferably, selected from the group consisting of skin protecting agents, skin conditioning agents, antioxidants, anti-acne agents, collagen promoters, soluble collagen, self tanner and mixtures thereof. More preferably, the skin care agent is selected from antiwrinkle agents, anti-skin-atrophy agents, antioxidants and anti-acne agents.

The skin protecting agents are agents that protect the skin against chemical irritants and/or physical irritants, e.g., UV light, including sunscreens, anti-acne additives, anti-wrinkle and anti-skin atrophy agents.

Suitable UV blocking agents include zinc oxide, titanium oxide, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4′-methoxy-t-butyldibenzoylmethane, 4-isopropy dibenzoylmethane, 3-benzylidene camphor, 3-(4-methylbenzylidene)camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane.

Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (e.g., glycolic acid and lactic acid), phytic acid, lipoic acid and lysophosphatidic acid.

The compositions of the invention, preferably, further comprise at least one emollient/humectant/moisturizer (e.g., butylene glycol) and/or at least one additional active ingredient, at least one cosmetically acceptable aesthetic component and/or at least one cosmetically acceptable excipient.

The at least one cosmetically acceptable vehicle or carrier is, preferably, a liquid, which is preferably water or a cosmetically acceptable aqueous buffer having a pH of about 7.0 to about 7.4, and, more preferably, the water or aqueous buffer is purified and/or sterile.

The at least one cosmetically acceptable excipient is, preferably, selected from the group consisting of surfactant/emulsifying agents, absorbents, antifoaming agents, binders, biological additives, chelating agents, denaturants, preservatives, solubilizing agents, solvents and thickening agents.

The at least one additional active ingredient is, preferably, selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, collagen promoters, exfoliators, self tanners, chelators, hair removers, anti-erythema agents, anti-redness agents, anti-rosacea agents, depuffing agents, anti-edema agents, anti-swelling agents, green tea extract, P. emblica (Amla), arnica, chamomile extract and cucumber extract. The at least one active ingredient is, preferably, at least one antioxidant, anti-acne agent, collagen promoter, soluble collagen, self tanner or mixtures thereof. More preferably, the at least one additional active ingredient is at least one antioxidant or anti-acne agent.

Suitable collagen promoters are peptides.

Suitable surfactant/emulsifying agents include ceteareths, ceteths, laneths, laureths, isoseareths, steareths, cetyl alcohol, deceths, dodoxynols, glyceryl palmitate, glyceryl stearate, laneths, myreths, nonoxynols, octoxynols, oleths, PEG-castor oil, poloxamers (e.g., poloxamer 407), poloxamines, polysorbates, sodium laurate, ammonium laureth sulfate, sodium laureth sulfate, sodium lauroyl sarcosinate, sodium lauroyl taurate, sodium lauryl sulfate, sodium methyl cocoyl taurate, sodium methyl oleoyl taurate, sodium nonoxynol sulfate, sodium cetyl sulfate, sodium cetearyl sulfate, sodium cocoate, sodium cocoyl isethionate and sodium cocoyl sarcosinate. Other suitable surfactant/emulsifying agents would be known to one of skill in the art and are listed in the CTFA International Cosmetic Ingredient Dictionary and Handbook, Vol. 2, 7th Edition (1997). Preferred surfactants include octoxynol-9 and polysorbate-20.

Examples of suitable preservatives include imidazolidinyl urea, diazolidinyl urea, phenoxyethanol, methylparaben, ethylparaben and propylparaben.

Examples of thickening agents include isopropyl myristate, isopropyl palmitate, isodecyl neopentanoate, squalene, mineral oil, C.sub.12-C.sub.15 benzoate and hydrogenated polyisobutene.

Examples of antioxidants and/or free-radical scavengers include ascorbic acid, salts of ascorbic acid such as ascorbyl palmitate and sodium ascorbate, ascorbyl glucosamine, vitamin E (i.e., tocopherols such as .alpha.-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), lipoic acid, sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), butylated hydroxyanisole (BHA), indole-3-carbinol, pycnogenol, melatonin, sulforaphane, pregnenolone, lipoic acid and 4-hydroxy-5-methyl-3 [2H]-furanone.

The antimicrobial agents can be antibacterial agents and/or antifungal agents. Examples of the antimicrobial agents include farnesol, benzoyl peroxide, erythromycin, tetracycline, triclosan, azelaic acid, clindamycin, chlorhexidine, neomycin, miconazole and clotrimazole.

The skin conditioning agents can be emollients, humectants and moisturizers, which include urea, guanidine, aloe vera, glycolic acid and glycolate salts such as ammonium and quaternary alkyl ammonium, lactic acid and lactate salts such as sodium lactate, ammonium lactate and quaternary alkyl ammonium lactate, polyhydroxy alcohols such as sorbitol, glycerol, hexanetriol, propylene glycol, butylene glycol, hexylene glycol, polyethylene glycol, carbohydrates such as alkoxylated glucose, starches, starch derivatives, glycerin, pyrrolidone carboxylic acid (PCA), lactamide monoethanolamine, acetamide monoethanolamine, volatile silicone oils, nonvolatile silicone oils, and mixtures thereof. Suitable silicone oils can be polydialkylsiloxanes, polydiarylsiloxanes, polyalkarylsiloxanes and cyclomethicones having 3 to 9 silicon atoms.

Skin soothing agents include bisabolol.

Suitable skin bleaching agents include, for example, hydroquinone, kojic acid and sodium metabisulfite.

The at least one aesthetic agent can be at least one of fragrances, pigments, colorants, essential oils, skin sensates and astringents. Examples of the suitable essential oils are olive oil, rose oil, palm oil, lavender oil, almond oil, Oenothera biennis (evening primrose) oil, clove oil, eucalyptus oil, peppermint oil and spearmint oil. Suitable aesthetic agents include clove oil, menthol, camphor, eucalyptus oil, eugenol, methyl lactate, bisabolol, witch hazel distillate and green tea extract.

The at least one anti-oxidant preferably is selected from ascorbic acid, ascorbyl palmitate, ascorbyl glucosamine, tocopheryl acetate, retinyl palmitate, superoxide dismutase, or mixtures thereof. The at least one skin conditioning agent preferably is selected from cyclomethicone and/or dimethiconol. The at least one reducing agent preferably is ubiquinone. The at least one additional active agent can be Camellia sinensis leaf extract and/or white tea extract or juice, preferably Camellia sinensis leaf extract. The at least one cosmetically acceptable vehicle or carrier can be purified or sterile water, preferably purified water. The at least one cosmetically acceptable excipient can be phospholipids.

The present invention provides compositions having the components listed in the following exemplary tables. It will be understood that the ingredient listings and weight percentages are only exemplary and are not intended to be limiting. Any of the ingredients discussed herein can be included in an amount between 0.001%. and 100%, as desired.

TABLE 1

| Ingredients | Weight Percent |
|---|---|
| coffee | 15% |
| turmeric powder | 20% |
| water | balance |

TABLE 2

| Ingredients | Weight Percent |
|---|---|
| coffee | 15% |
| turmeric powder | 20% |
| noni juice (Citrifolla Morinda) | 25% |
| aloe vera juice (Aloe barbadensis) | 25% |
| water | balance |

TABLE 3

| Ingredients | Weight Percent |
|---|---|
| coffee | 15% |
| turmeric powder | 20% |
| noni juice (Citrifolla Morinda) | 25% |
| aloe vera juice (Aloe barbadensis) | 25% |
| gingko biloba | 2% |
| water | balance |

TABLE 4

| Ingredients | Weight Percent |
|---|---|
| coffee | 15% |
| turmeric powder | 20% |
| noni juice (Citrifolla Morinda) | 25% |
| aloe vera juice (Aloe barbadensis) | 25% |
| gingko biloba | 2% |
| Camellia sinensis tea | 5% |
| water | balance |

TABLE 5

| Ingredients | Weight Percent |
|---|---|
| coffee | 15% |
| turmeric powder | 20% |
| noni juice (Citrifolla Morinda) | 25% |
| aloe vera juice (Aloe barbadensis) | 25% |
| Gingko biloba | 2% |
| Camellia sinensis tea | 5% |
| oil (e.g., grapeseed oil (Vitis vinifera), | 4% |
| jojoba oil (Simmondsia chinensis), coconut | |
| oil (Cocos nucifera), neem oil (Azadirachta | |
| indica), Kukui nut oil (Aleurites | |
| moluccana), Macadamia nut oil | |
| (Macadamia integrifolia)) | |
| water | balance |

TABLE 6

| Ingredients | Weight Percent |
|---|---|
| coffee | 15% |
| turmeric powder | 20% |
| noni juice (Citrifolla Morinda) | 25% |
| aloe vera juice (Aloe barbadensis) | 25% |
| gingko biloba | 2% |
| Camellia sinensis tea | 4% |
| oil | 4% |
| (e.g., grapeseed oil (Vitis vinifera), jojoba | |
| oil (Simmondsia chinensis), coconut oil | |
| (Cocos nucifera), neem oil (Azadirachta | |
| indica), Kukui nut oil (Aleurites | |
| moluccana), Macadamia nut oil | |
| (Macadamia integrifolia)) | |
| juice | 4% |
| (e.g., acai juice (Euterpe oleracea), | |
| blueberry juice (any in the Vaccinium | |
| genus), cranberry juice (Vaccinium | |
| oxycoccos), mangosteen juice (Garcinia | |
| mangostana), pomegranate juice (Punica | |
| granatum)) | |
| water | balance |

TABLE 7

| Ingredients | Weight Percent |
|---|---|
| coffee | 15% |
| turmeric powder | 20% |
| noni juice (Citrifolla Morinda) | 20% |
| aloe vera juice (Aloe barbadensis) | 20% |
| gingko biloba | 2% |
| Camellia sinensis tea | 5% |
| oil | 4% |
| (e.g., grapeseed oil (Vitis vinifera), jojoba | |
| oil (Simmondsia chinensis), coconut oil | |
| (Cocos nucifera), neem oil (Azadirachta | |
| indica), Kukui nut oil (Aleurites | |
| moluccana), Macadamia nut oil | |
| (Macadamia integrifolia)) | |
| juice | 4% |
| (e.g., acai juice (Euterpe oleracea), | |
| blueberry juice (any in the Vaccinium | |
| genus), cranberry juice (Vaccinium | |
| oxycoccos), mangosteen juice (Garcinia | |
| mangostana), pomegranate juice (Punica | |
| granatum)) | |
| honey | 5% |
| water | balance |

TABLE 8

| Ingredients | Weight Percent |
|---|---|
| coffee | 15% |
| turmeric powder | 20% |
| noni juice (Citrifolla Morinda) | 20% |
| aloe vera juice (Aloe barbadensis) | 20% |
| gingko biloba | 2% |
| Camellia sinensis tea | 5% |
| oil | 4% |
| (e.g., grapeseed oil (Vitis vinifera), jojoba | |
| oil (Simmondsia chinensis), coconut oil | |
| (Cocos nucifera), neem oil (Azadirachta | |
| indica), Kukui nut oil (Aleurites | |
| moluccana), Macadamia nut oil | |
| (Macadamia integrifolia)) | |
| fruit juice | 4% |
| (e.g., acai juice (Euterpe oleracea), | |
| blueberry juice (any in the Vaccinium | |
| genus), cranberry juice (Vaccinium | |
| oxycoccos), mangosteen juice (Garcinia | |
| mangostana), or pomegranate juice (Punica | |
| granatum)) | |
| honey | 5% |
| zinc oxide or titanium oxide | 4% |
| water | balance |

Set forth below are exemplary uses of the compositions of the present invention.

### Example 1

The subject was an 82 year old human male with Alzheimer's Disease living in a Veterans' hospital for several years. Before treatment, the subject's condition had deteriorated to the point that he had not spoken to anyone for five to six weeks. Prior to that, although he was able to converse, it was generally unintelligible. The subject also had eczema with dry, flaky, cracked skin on his arms. Formulation D (described above) was applied topically to the subject's skin condition over a two week period of time. After two weeks, the skin condition showed vast improvement. Also, the subject's son was able to converse with the subject several times in an intelligible manner for the first time in years. The subject generally showed improvement over his previous state. Specifically, the subject was able to hold a 30 minute conversation with his son during which he spoke in complete sentences and asked if he could attend his upcoming grandchild's wedding, indicating comprehension and making plans for a future event.

### Example 2

The subject was a 57 year old human male with a history of squamous and basil cell skin cancers. The formulation included coffee, green tea, honey, grape seed oil and turmeric powder. The ingredients was mixed, shaken and applied with either a paper towel and let set for 10-30 minutes and then washed off with shampoo or was applied on a circular bandage and left on for several hours. The formulation comprised 2 ounces of brewed coffee, 2 ounces of brewed green tea, 1 tablespoon of honey, 2 ounces of grapeseed oil and 3 ounces of turmeric powder. It was applied as described above topically to pre-cancerous skin lesions twice a day. The skin lesions absorbed the composition and were stained yellow, as opposed to the regular skin which did not absorb the stain. Within a few days or weeks depending on the severity of the lesions, the lesions fell off and did not reoccur.

The same treatment has also been used with coconut oil, noni juice, aloe vera juice and honey added to the formulation.

### Example 3

A mixture was made using the following ingredients: 2 ounces of brewed coffee, 2 ounces of brewed green tea, 2 ounces of coconut oil, 2 ounces of noni juice, 2 ounces of aloe vera juice, 3 ounces of honey, 2 ounces of grapeseed oil and 3 ounces of turmeric powder. The ingredients were added in the above order along with stiffing in the coffee and tea before adding coconut oil and honey. The honey and coconut oil were heated to help with the mixing process. Stir and then shake. The same 57 year old male subject as example 2 applied the mixture using a paper towel for 15 minutes daily. The composition appeared to work even faster than that of Example 3.

### Example 4

A mixture was made using the following ingredients: 2 ounces of instant coffee, 2 ounces of instant tea, 2 ounces of noni juice, 2 ounces of aloe vera juice, 1 ounce of grape seed oil, 1 ounce of coconut oil, 1 ounce of jojoba oil and 4 ounces of honey. A jar of sunscreen was made using 1 oz. of the above mixture combined with 1 teaspoon of turmeric powder, 240 mg ginkgo biloba, and 2 ounces of zinc oxide ointment. Other than the zinc oxide, the ingredients were all mixed in a jar then stirred and shaken until a relatively uniform consistency was realized. The zinc oxide ointment was then added. The formulation was heated in a microwave for 10 seconds, stirred and heated again. A lid was then put on the jar and it was shaken vigorously for 1 minute, after which it was placed in the refrigerator immediately for 1 hour.

The same 57 year old male subject as examples 2-3 was diagnosed with a squamous cell cancer lesion. The doctor told the subject it would need to be excised. It was about 0.5 inches in diameter and raised about 0.25 inches off of the skin. Before the excision appointment, the mixture was applied on a bandage several times a week. In one month the lesion fell off. When the subject went in for the excision, the lab report showed no signs of cancer lesions and an abnormal amount of freckling. This same application process has worked for other cancerous legions on the subject in as little as one to two weeks.

### Example 5

In another exemplary formulation, the following ingredients were combined in a jar: 2 ounces of noni juice, 2 ounces of aloe vera juice, 1 ounce of grapeseed oil, 1 ounce of jojoba oil, 1 ounce of coconut oil, 4 ounces of honey, 2 ounces of instant coffee (Folger's® Crystals), 2 ounces of instant tea (preferably green, but may be black), 1 ounce of neem oil, 1 ounce of pomegranate juice, 1 ounce of cranberry juice and 1 ounce of blueberry juice. The ingredients were added in the order set forth above. The honey and coconut oil were heated in a microwave to get a runny liquid and the coffee and tea were mixed in last. The ingredients were stirred and then shaken for 2 minutes to create a “mixture”.

To create the formulation, 1 ounce of the mixture was combined with 2 oz. of zinc oxide ointment, 1 teaspoon of turmeric powder, 240 mg of gingko biloba, and ½ teaspoon of zinc oxide powder. The jar was then shaken vigorously until blended and then immediately refrigerated.

The same 57 year old male as examples 2-4 found favorable results as an all-natural sunscreen that was non-slippery, insect repellant, and removed skin cancers and treated other dermatological conditions as discussed above.

### Example 6

A formulation was made for use with a 14 year old dog. The formulation included 1 ounce of grapeseed oil, 1 ounce of jojoba oil, 1½ ounces of turmeric powder, 720 mg. of gingko biloba, 2 ounces of instant coffee and 2 ounces of instant tea. The powder ingredients (turmeric powder, ginko biloba, instant coffee, and instant tea) were dissolved in the grapeseed oil and jojoba oil while being heated. The remaining ingredients of 2 ounces of noni juice, 2 ounces of aloe vera juice, and 4 ounces of honey were mixed in to create a “mixture”. This mixture can be stored for use either as an oral version for a tonic, a spray, or just an easier way to mix the composition with the zinc oxide ointment, aloe vera gel, or just about any other ingredient as described above.

A ½ ounce of the “mixture” was mixed with 1½ ounces of water in a spray bottle to treat a large rash skin irritation on the subject dog's belly. The rash was gone the next day.

The 14 year old, lethargic dog also began taking the composition orally. Before treatment, the dog was unable to climb stairs or get in the car and had a large tumor the size of a volleyball in his abdomen. The subject was given the formulation orally in four doses over a two week period of time. Two weeks after the initial administration the dog was frisky, running, barking, playing and jumping on the couch to get his ball. The tumor was reduced in size by about 25% after two weeks.

In other uses, the inventor has found that the inventive compositions and formulations treat osteoarthritis. A subject rubbed Formulation D on a swollen and sore neck. It reduced swelling and pain. After three treatments the neck pain that had lasted prior to treatment for about 4 months disappeared and did not return even after discontinuing treatment.

The compositions work as an anti-inflammatory so they can be combined with medicines that cause irritation (like Carac or chemotherapy). The formulations can also be diluted considerably so that 1 ounce of the Formula F or G in a tub of bath water could be a treatment for many ailments since it absorbs through the skin. The formulations can be added to improve almost any cosmetic or medicinal product. Therefore, the inventive compositions and formulations can be used topically as a lotion, spray, gel, ointment, salve, bath soak or with a skin patch that can be left on continuously to treat any of the above conditions. They can be applied rectally to treat hemorrhoids or polyps. They can also be formulated for oral use.

In another embodiment, Formulation F or any of the other formulations can be mixed with a substance used for contrast imaging in radiology such as barium or radioactive iodine because of the propensity for the compositions or formulations to be attracted to abnormal tissue and pass over healthy tissue. The mixture could be used to scan for internal tumors to help diagnosis.

The inventor has found that by using the composition disclosed herein wounds heal faster; skin rashes go away faster; swelling, pain and itching from skin sores is reduced; cold sores are covered up and heal faster; boils, pimples, and sebaceous cysts heal faster; polyps turn black and fall off; squamous cell and basil cell skin cancers turn yellow and with repeated treatment (overnight with a bandage for 2-4 weeks) fall off; eczema subsides or is prevented; hemorrhoids stop hurting almost immediately upon treatment; heel cracks disappear in a few days, scar tissue reduces in size and color, actinic keratosis or pre-cancers turn yellow and do not convert to skin cancer, and reduce in size and disappear or fall off completely; and hot spots on dogs heal faster, often within one day.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A composition comprising from about 0.01% to about 40% by weight of a member of the curcuma genus, from about 0.01% to about 40% by weight of a member of the coffea genus and the balance being a pharmaceutically acceptable carrier.

2. The composition of claim 1 wherein the member of the curcuma genus constitutes between about 2% and about 15% by weight of the total weight of the composition.

3. The composition of claim 1 wherein the member of the coffea genus constitutes between about 2% and about 20% by weight of the total weight of the composition.

4. The composition of any one of claims 1-3 wherein the member of the curcuma genus is turmeric and the member of the coffea genus is coffea arabica.

5. The composition of any one of claims 1-4 further comprising from about 0.1% to about 50% noni juice (Citrifolla Morinda), and about 0.1% to about 50% aloe vera juice (Aloe barbadensis).

6. The composition of claim 5 wherein the noni juice (Citrifolla Morinda) constitutes between about 4% and about 35% by weight of the total weight of the composition.

7. The composition of claim 5 wherein the aloe vera juice (Aloe barbadensis) constitutes between about 4% and about 35% by weight of the total weight of the composition.

8. The composition of any one of claims 1-7 further comprising from about 0.001% to about 5% by weight gingko biloba.

9. The composition of claim 8 wherein the gingko biloba constitutes between about 0.2% and about 2% by weight of the total weight of the composition.

10. The composition of any one of claims 1-9 further comprising from about 0.01% to about 40% Camellia sinensis tea.

11. The composition of claim 10 wherein the Camellia sinensis tea constitutes between about 0.2% and about 20% by weight of the total weight of the composition.

12. The composition of claim 10 or 11 wherein the Camellia sinensis tea is selected from the group white tea, yellow tea, green tea, black tea, oolong tea or Pu-erh tea.

13. The composition of any one of claims 1-12 where the coffee is instant coffee.

14. The composition of any one of claims 10-12 where the coffee is instant coffee and the tea is instant tea.

15. The composition of any one of claims 1-14 further comprising from about 0.1% to about 50% of one or more of the following oils: grapeseed oil (Vitis vinifera), jojoba oil (Simmondsia chinensis), coconut oil (Cocos nucifera), neem oil (Azadirachta indica), kukui nut oil (Aleurites moluccana), macadamia nut oil (Macadamia integrifolia).

16. The composition of claim 15 wherein the oil constitutes between about 4% and about 35% by weight of the total weight of the composition.

17. The composition of any one of claims 1-16 further comprising from about 0.1% to about 50% of one or more of the following juices: acai juice (Euterpe oleracea), blueberry juice (any in the Vaccinium genus), cranberry juice (Vaccinium oxycoccos), mangosteen juice (Garcinia mangostana), pomegranate juice (Punica granatum).

18. The composition of claim 17 wherein the juice constitutes between about 4% and about 75% by weight of the total weight of the composition.

19. The composition of any one of claims 1-18 further comprising from about 0.1% to about 60% honey.

20. The composition of claim 19 wherein the honey constitutes between about 5% and about 45% by weight of the total weight of the composition.

21. The composition of any one of claims 1-20 further comprising a UV blocking agent.

22. The composition of any of claims 1-21 further comprising a moisturizing agent.

23. The composition of any of claims 1-22 further comprising an enhancer.

24. The composition of any of claims 1-23 further comprising a skin care agent.

25. The composition of any one of claims 1-24 wherein application of the composition to the skin of an animal improves one selected from the following maladies skin cancer and pre-cancers, hemorrhoids, rashes, wounds, scars, age spots, polyps, swollen joints, inflammation, cracked skin, heel cracks, ulcerated skin, bedsores, saddle sores, hot spots, insect bites, shingles, herpes, eczema, and psoriasis.

26. A method of treating a condition selected from the group consisting of dry skin, dandruff, warts, acne, sebaceous cysts, wounds, rashes, shingles, herpes/cold sores, boils, pimples, polyps, skin cancers, hemorrhoids, scars, psoriasis, eczema, pruritus, age spots, reduced skin moisture, spider veins, senile purpura, lentigines, melasmas, deepening of skin lines, blotches, wrinkles, blemished skin, nodules, atrophy, rosacea, impetigo, actinic keratosis precancerous lesions, telangiecatic skin, hyperpigmented skin, hyperkeratotic skin, nail infections, inflammatory dermatoses, tumors, cancers, Alzheimer's disease, arthritis, lupus, inflammation caused by shaving, swelling, bumps or bruises on the skin, insect and spider bites, cracked skin, heel cracks, ulcerated skin, swollen joints, inflammation, the method comprising the step of administering to a human or animal a composition comprising from about 0.01% to about 40% by weight of a member of the curcuma genus, from about 0.01% to about 40% by weight of a member of the coffea genus and the balance being a pharmaceutically acceptable carrier.
